(19) United States
(12) Patent Application Publication
(10) Pub. No.: US 2021/0090255 A1
(43) Pub. Date: Mar. 25, 2021

(54) RECORDING MEDIUM RECORDING PROGRAM, INFORMATION PROCESSING APPARATUS, AND INFORMATION PROCESSING METHOD

(71) Applicant: FUJITSU LIMITED, Kawasaki-shi (JP)

(72) Inventors: Takashi Kumagai, Sakata (JP); Tadashi Fujioka, Kakogawa (JP); Chikashi Kigure, Kawasaki (JP); Kazuhiko Katayama, Sumida (JP); Yuji Deki, Ota (JP); Shirin Aoki, Ota (JP); Fumiyuki Takehisa, Yokohama (JP); Tsugumasa Yamamoto, Yokohama (JP); Wataru Oura, Yokohama (JP)

(73) Assignee: FUJITSU LIMITED, Kawasaki-shi (JP)

(21) Appl. No.: 17/114,806

(22) Filed: Dec. 8, 2020

Related U.S. Application Data
(63) Continuation of application No. PCT/JP2018/022620, filed on Jun. 13, 2018.

(51) Int. Cl. G06T 7/00; G06F 3/0481; A61B 6/14

(52) U.S. Cl. CPC G06T 7/0012; G06F 3/04812; A61B 6/14; G06T 2207/10116; G06T 2200/24; G06T 2207/20081; G06T 2207/30036

(57) ABSTRACT

A recording medium stores therein a program for causing a computer to execute processing includes: acquiring images of N or less each including teeth adjacent to each other; arranging and displaying, in N image display areas capable of displaying the images in association with an arrangement of teeth, each of the images, in association with each of the N image display areas, a selection area for selecting presence or absence of an image and input areas for inputting information indicating a condition of each tooth included in an image displayed in each of the N image display areas; receiving a selection content in the selection area or input contents in the input areas or combination of the selection content and the input contents; and generating learning information in which a condition of each of the teeth and an image of each of the teeth are associated with each other.

### CROSS-REFERENCE TO RELATED APPLICATION

This application is a continuation application of International Application PCT/JP2018/022620 filed on Jun. 13, 2018 and designated the U.S., the entire contents of which are incorporated herein by reference.

### FIELD

The embodiment discussed herein is related to a program, an information processing apparatus, and an information processing method.

### BACKGROUND

Conventionally, artificial intelligence (AI) diagnosis based on X-ray images and computed tomography (CT) images has been performed. The AI diagnosis is performed, for example, by using a large number of images of a specific organ as learning objects to learn what kind of image features indicate that an organ to be diagnosed is normal (or indicate a disease in the organ).

Japanese Laid-open Patent. Publication No. 2012-150801, International Publication Pamphlet No. WO 2015/141760, and Japanese Laid-open Patent Publication No. 2017-035173 are disclosed as related art.

### SUMMARY

According to an aspect of the embodiments, a non-transitory computer-readable recording medium stores therein a program for causing a computer to execute processing includes: acquiring a plurality of images of N or less each including a plurality of teeth adjacent to each other; arranging and displaying, in N image display areas capable of displaying the plurality of images in association with an arrangement of teeth, each of the plurality of images in corresponding one of the image display areas; displaying, in association with each of the N image display areas, a selection area for selecting presence or absence of an image and a plurality of input areas for inputting information indicating a condition of each tooth included in an image displayed in each of the N image display areas; receiving a selection content in the selection area or input contents in the plurality of input areas or combination of the selection content and the input contents, corresponding to each of the N image display areas; and generating learning information in which a condition of each of the plurality of teeth and an image of each of the plurality of teeth are associated with each other in a state capable of identifying positions of the plurality of teeth, on the basis of the received selection content and input contents and the plurality of images that has been registered.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a block diagram schematically illustrating a configuration example of an information processing system according to an embodiment;

FIG. 2 is a block diagram schematically illustrating a hardware configuration example of an information processing apparatus and a terminal illustrated in FIG. 1;

FIG. 3A is a block diagram schematically illustrating a functional configuration example of the information processing apparatus illustrated in FIG. 2;

FIG. 3B is a block diagram schematically illustrating a functional configuration example of the terminal illustrated in FIG. 2;

FIG. 4 is a diagram illustrating a first screen display example in the terminal illustrated in FIG. 2;

FIG. 5 is a diagram illustrating a second screen display example in the terminal illustrated in FIG. 2;

FIGS. 6A to 6C are diagrams illustrating a configuration example of storage data in the terminal illustrated in FIG. 2;

FIGS. 7A and 7B are diagrams illustrating a configuration example of temporary storage data in the terminal illustrated in FIG. 2;

FIGS. 8A and 8B are diagrams illustrating a configuration example of image data for which evaluation results are not input in the terminal illustrated in FIG. 2;

FIGS. 9A and 9B are diagrams illustrating a configuration example of all stored image data in the terminal illustrated in FIG. 2;

FIGS. 10A and 10B are diagrams illustrating a configuration example of stored patient-specific image data in the terminal illustrated in FIG. 2;

FIG. 11 is a flowchart illustrating learning information generation processing in the information processing apparatus illustrated in FIG. 2; and

FIG. 12 is a flowchart illustrating details of input reception processing illustrated in FIG. 11.

### DESCRIPTION OF EMBODIMENTS

For example, by applying image diagnosis to an oral cavity image (for example, “X-ray image of a tooth”), a condition of a tooth may be diagnosed. In this case, for example, X-ray images of teeth are used as learning objects to learn what kind of image features indicate that a tooth to be diagnosed is normal (or abnormal).

Since the shape of a tooth differs depending on whether the tooth is a molar, a front tooth, an upper tooth, or a lower tooth, it is assumed that image features of each tooth are learned after a position of each tooth is identified.

Thus, after the position of each tooth is identified so that a tooth at the same position may be identified in any patient (for example, “normalized”), learning data in which a condition of a tooth to be diagnosed and an image are associated with each other is prepared.

However, it is not efficient for an operator such as a dentist to label a condition of a tooth while inputting an identifier of a position of the tooth in order to prepare learning data.

In one aspect, learning data for automatic diagnosis of a condition of an oral cavity may be efficiently generated.

Hereinafter, an embodiment will be described with reference to the drawings. Note that the embodiment to be described below is merely an example, and there is no intention to exclude application of various modifications and techniques not explicitly illustrated in the embodiment. The present embodiment may be modified in a various ways to be implemented without departing from the spirit thereof.

Furthermore, each drawing is not intended to include only components illustrated in the drawing, and may include other functions and the like.

Hereinafter, parts denoted by the same reference numerals indicate similar parts in the drawings.

[A] Example of Embodiment

[A-1] System Configuration Example

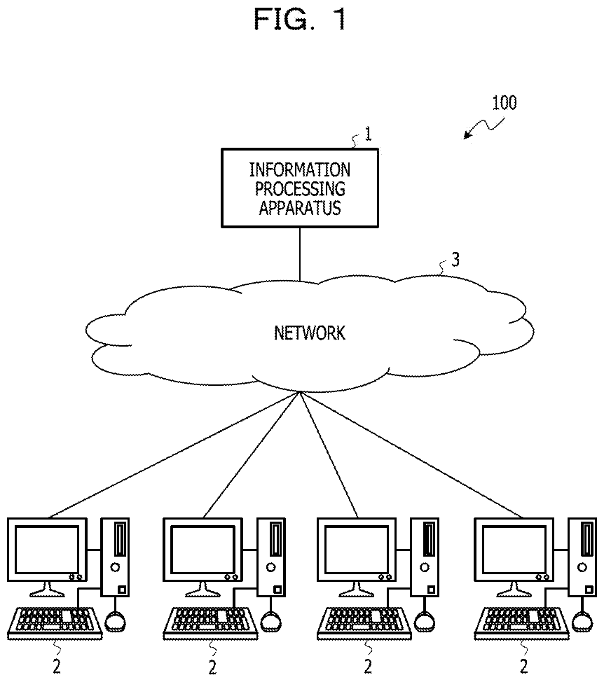

FIG. 1 is a block diagram schematically illustrating a configuration example of an information processing system 100 according to the embodiment.

The information processing system 100 includes an information processing apparatus 1 and a plurality of (four in the illustrated example) terminals 2. The information processing apparatus 1 and the plurality of terminals 2 are communicably connected to each other via a network 3.

The information processing apparatus 1 is a computer having a server function, and provides a dental X-ray image evaluation tool to each of the plurality of terminals 2.

The plurality of terminals 2 is arranged in, for example, a dental clinic or a hospital. The plurality of terminals 2 receives input of various kinds of information from a dentist or a dental hygienist (may be referred to as a “user”), and also displays various kinds of information for a dentist or a dental hygienist.

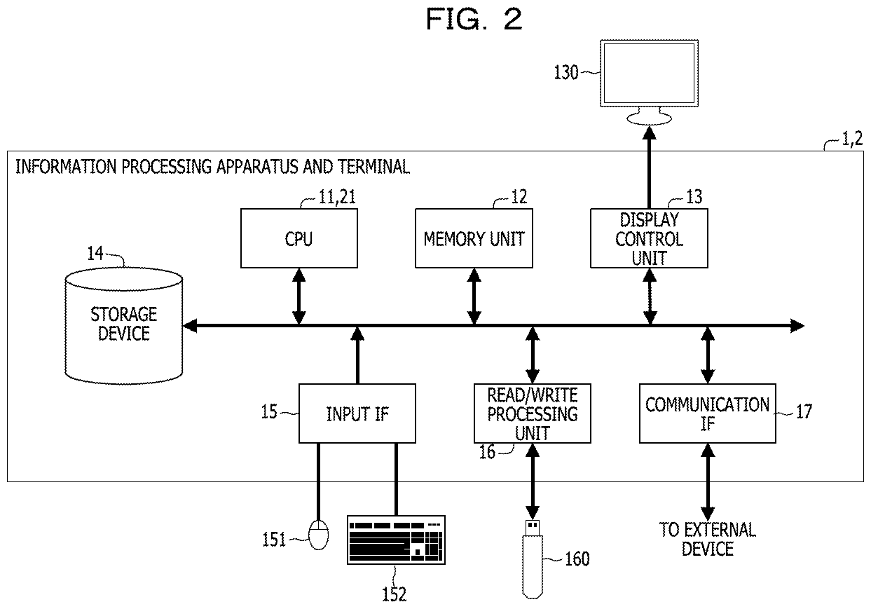

FIG. 2 is a block diagram schematically illustrating a hardware configuration example of the information processing apparatus 1 and the terminal 2 illustrated in FIG. 1.

The information processing apparatus 1 includes a central processing unit (CPU) 11, a memory 12, a display control unit 13, a storage device 14, an input interface (I/F) 15, a read/write processing unit 16, and a communication I/F 17. Furthermore, the terminal 2 includes a CPU 21, a memory 12, a display control unit 13, a storage device 14, an input I/F 15, a read/write processing unit 16, and a communication I/F 17.

The memory 12 is, for example, a storage device including a read only memory (ROM) and a random access memory (RAM). In the ROM of the memory 12, programs such as a basic input/output system (BIOS) may be written. A software program of the memory 12 may be appropriately read and executed by the CPU 11 or 21. Furthermore, the RAM of the memory 12 may be used as a primary recording memory or a working memory.

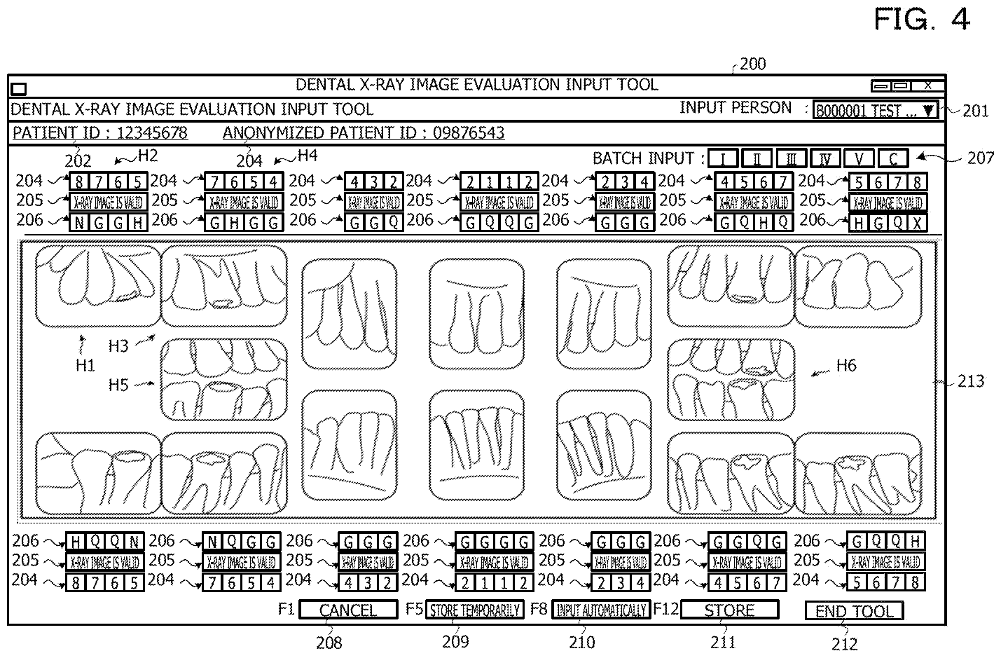

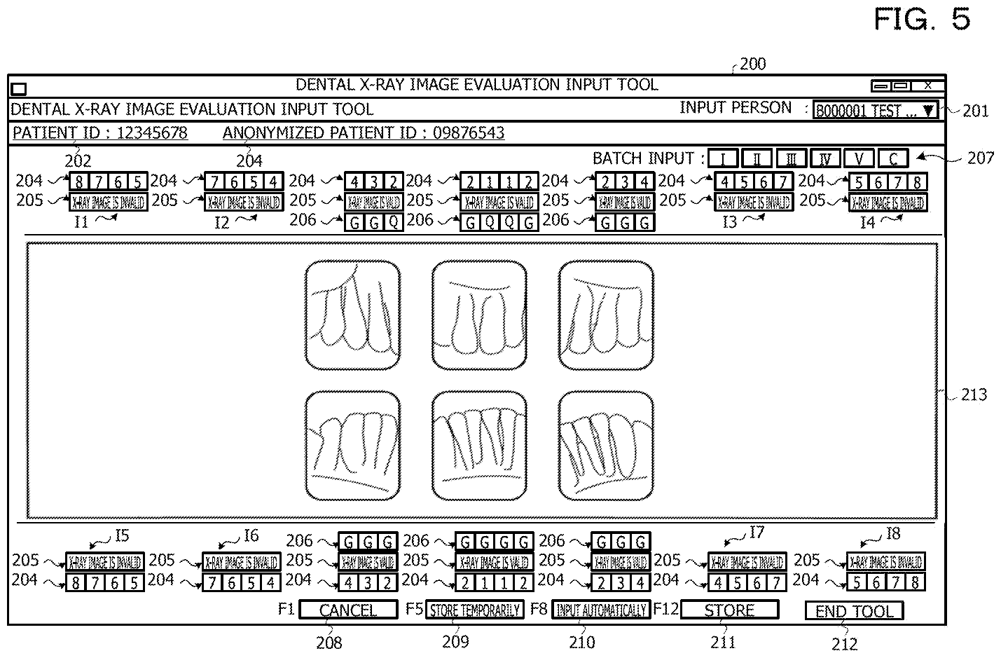

The display control unit 13 is connected to a display device 130, and controls the display device 130. The display device 130 is a liquid crystal display, an organic light-emitting diode (OLED) display, a cathode ray tube (CRT), an electronic paper display, or the like, and displays various kinds of information for an operator or the like. The display device 130 may be combined with an input device, and may be, for example, a touch panel. On the display device 130 of the terminal 2, an input screen 200 for evaluation results of a dental X-ray image, which will be described later with reference to FIGS. 4 and 5, is displayed.

The storage device 14 is, for example, a device that stores data in a readable and writable manner, and for example, a hard disk drive (HDD), a solid state drive (SSD), and a storage class memory (SCM) may be used.

The input I/F 15 is connected to an input device such as a mouse 151 and a keyboard 152, and controls the input device such as the mouse 151 and the keyboard 152. The mouse 151 and the keyboard 152 are examples of the input devices, and an operator performs various kinds of input operation through these input devices.

The read/write processing unit 16 is configured so that a recording medium 160 may be attached thereto. The read/write processing unit 16 is configured to be capable of reading information recorded in the recording medium 160 when the recording medium 160 is attached thereto. In the present example, the recording medium 160 is portable. For example, the recording medium 160 is a flexible disk, an optical disc, a magnetic disc, a magneto-optical disc, a semiconductor memory, or the like.

The communication I/F 17 is an interface for enabling communication with an external device. The information processing apparatus 1 is communicably connected to the plurality of terminals 2 via the communication I/F 17. Furthermore, each terminal 2 is communicably connected to the information processing apparatus 1 via the communication IF 17.

Each of the CPUs 11 and 21 is a processing device that performs various kinds of control and calculation, and implements various functions by executing an operating system (OS) and programs stored in the memory 12.

The CPU 11 controls, for example, operation of the entire information processing apparatus 1, and the CPU 21 controls, for example, the entire terminal 2. The device for controlling operation of the entire information processing apparatus 1 and the entire terminal 2 is not limited to the CPUs 11 and 21, and may be any one of an MPU, DSP, ASIC, PLD, and FPGA, for example. Furthermore, the device for controlling operation of the entire information processing apparatus 1 and the entire terminal 2 may be a combination of two or more of the CPU, MPU, DSP, ASIC, PLD, and FPGA. Note that the MPU is an abbreviation for a micro processing unit, the DSP is an abbreviation for a digital signal processor, and the ASIC is an abbreviation for an application specific integrated circuit. Furthermore, the PLD is an abbreviation for a programmable logic device, and the FPGA is an abbreviation for a field programmable gate array.

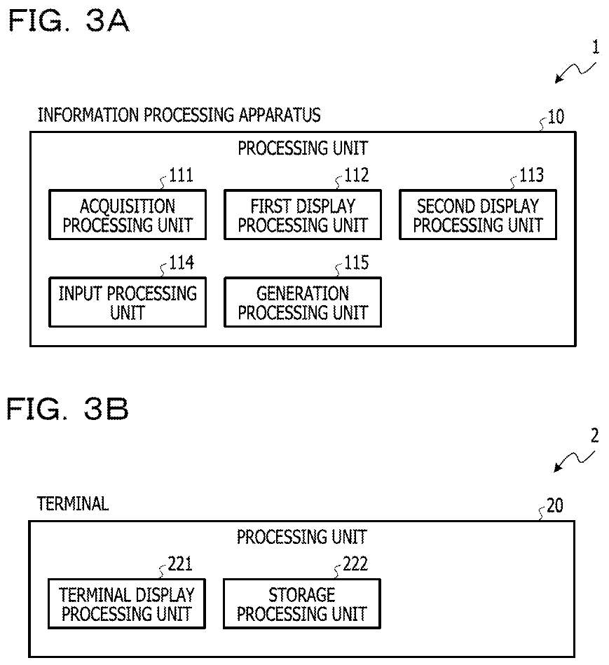

FIG. 3A is a block diagram schematically illustrating a functional configuration example of the information processing apparatus 1 illustrated in FIG. 2. Furthermore, FIG. 3B is a block diagram schematically illustrating a functional configuration example of the terminal 2 illustrated in FIG. 2.

The information processing apparatus 1 has a function as a processing unit 10. As illustrated in FIG. 3A, the processing unit 10 functions as an acquisition processing unit 111, a first display processing unit 112, a second display processing unit 113, an input processing unit 114, and a generation processing unit 115. Furthermore, the terminal 2 has a function as a processing unit 20. As illustrated in FIG. 3B, the processing unit 20 functions as a terminal display processing unit 221 and a storage processing unit 222.

Note that a program for implementing the functions as the acquisition processing unit 111, the first display processing unit 112, the second display processing unit 113, the input processing unit 114, the generation processing unit 115, the terminal display processing unit 221, and the storage processing unit 222 is provided, for example, in the form recorded in the aforementioned recording medium 160. Then, the computer reads the program from the recording medium 160 via the read/write processing unit 16, transfers the program to an internal storage device or an external storage device, and stores the program for use. Furthermore, for example, the program may be recorded in a storage device (recording medium) such as a magnetic disc, an optical disc, or a magneto-optical disc, and provided from the storage device to the computer via a communication path.

When the functions as the acquisition processing unit 111, the first display processing unit 112, the second display processing unit 113, the input processing unit 114, the generation processing unit 115, the terminal display processing unit 221, and the storage processing unit 222 are implemented, the program stored in the internal storage device is executed by a microprocessor of the computer. At this time, the computer may read and execute the program recorded in the recording medium 160. Note that, in the present embodiment, the internal storage device is the memory 12 and the microprocessor is the CPU 11 or 21.

On the basis of an instruction from the information processing apparatus 1, the terminal display processing unit 221 illustrated in FIG. 3B causes the display device 130 to display the input screen 200 for evaluation results of a dental X-ray image to be described later with reference to FIGS. 4 and 5.

The storage processing unit 222 illustrated in FIG. 3B stores, in the storage device 14, storage data, temporary storage data, image data for which evaluation results are not input, all image data, and patient-specific image data, which will be described later with reference to FIGS. 4 to 10.

The acquisition processing unit 111 illustrated in FIG. 3A receives registration of a plurality of X-ray images from the terminal 2. For example, the acquisition processing unit 111 acquires a plurality of images of N or less each including a plurality of teeth adjacent to each other. Note that N is a natural number of 3 or more.

The first display processing unit 112 illustrated in FIG. 3A displays, on the input screen 200 (to be described later with reference to FIGS. 4 and 5) of the terminal 2, a plurality of X-ray images whose registration has been received by the acquisition processing unit 111. For example, in N image display areas 213 (to be described later with reference to FIGS. 4 and 5) capable of displaying a plurality of images in association with an arrangement of teeth, the first display processing unit 112 arranges and displays each of the plurality of images in the corresponding one of the image display areas 213.

As described later with reference to FIGS. 4 and 5, the second display processing unit 113 illustrated in FIG. 3A displays, on the input screen 200 of the terminal 2, a selection area 205 for selecting whether an X-ray image has been registered and a plurality of input areas 206 for inputting information indicating a condition of each tooth. For example, the second display processing unit 113 displays, in association with each of the N image display areas 213, the selection area 205 for selecting presence or absence of an image, and the plurality of input areas 206 for inputting information indicating a condition of each tooth included in an image displayed in each of the N image display areas 213.

The input processing unit 114 illustrated in FIG. 3A receives, from the terminal 2, a selection content in the selection area 205 and input contents in the input areas 206. For example, the input processing unit 114 receives at least one of the selection content in the selection area 205 and the input contents in the plurality of input areas 206, corresponding to each of the N image display areas 213.

The generation processing unit 115 illustrated in FIG. 3A generates learning data on the basis of a selection content in the selection area 205, input contents in the input areas 206, and an X-ray image. For example, the generation processing unit 115 generates learning information in which a condition of each of a plurality of teeth and an image of each of the plurality of teeth are associated with each other in a state where positions of the plurality of teeth may be identified, on the basis of received selection contents and input contents, and registered plurality of images.

Furthermore, the generation processing unit 115 uses the generated learning information to generate, by deep learning, a learning model which corresponds to each of a plurality of teeth and relates to characteristics regarding a condition of each tooth. Moreover, the input processing unit 114 uses the generated learning model to generate information indicating a condition of each of a plurality of teeth included in a new image. Then, the second display processing unit 113 outputs the information indicating the condition of each of the plurality of teeth included in the new image.

The learning model may be generated, for example, by cutting a plurality of teeth included in a plurality of X-ray images one by one, including an alveolar bone, and associating an image of the cut tooth, a position of the tooth, and an evaluation result of the tooth. The cutting of an X-ray image may be automatically performed by learning a manually cut X-ray image.

FIG. 4 is a diagram illustrating a first screen display example in the terminal 2 illustrated in FIG. 2.

The input screen 200 for evaluation results of a dental X-ray image includes, for example, an input person selection area 201, a patient ID display area 202, an anonymized patient ID display area 203, a plurality of tooth number display areas 204, a plurality of the X-ray image presence/absence selection areas 205, and the plurality of evaluation result input areas 206. Furthermore, the input screen 200 includes, for example, a batch input button 207, a cancel button 208, a temporary storage button 209, an automatic input button 210, a storage button 211, a tool end button 212, and an image display area group 213. Note that the ID is an abbreviation for an identifier.

In the input person selection area 201, for example, the name of a dentist or a dental hygienist who inputs evaluation results is selected by a pull-down method. With this configuration, in a case where an incorrect evaluation result is input, a user who made the incorrect input may be specified.

Evaluation results input by a dentist or a dental hygienist may be approved only by the dentist or another dentist. Furthermore, when the name of a dentist or a dental hygienist is selected, authentication information such as an ID and a password may be requested to be input.

In the patient ID display area 202, a patient ID for uniquely identifying each patient is displayed in a dental clinic or a hospital where the terminal 2 is installed. The patient ID is held only in the corresponding terminal 2 and need not be held in the information processing apparatus 1.

In the anonymized patient ID display area 203, an anonymized patient ID obtained by converting a patient ID in order to anonymize an X-ray image and evaluation results of each patient in the information processing system 100 is displayed. Various algorithms may be used to convert the patient ID to the anonymized patient ID.

In the tooth number display areas 204, numbers of teeth that appear in a corresponding X-ray image are displayed.

In the input screen 200 illustrated in FIG. 4, in an upper left first image of the image display area group 213 (see a reference numeral H1), an upper right tooth numbered 8, an upper right tooth numbered 7, an upper right tooth numbered 6, and an upper right tooth numbered 5 appear, in this order from the left. Accordingly, in the tooth number display areas 204 (see a reference numeral H2) displayed above the upper left first image of the image display area group 213, the numbers 8, 7, 6, and 5 are displayed in this order from the left.

Furthermore, in the input screen 200 illustrated in FIG. 4, in an upper left second image of the image display area group 213 (see a reference numeral H3), the upper right tooth numbered 7, the upper right tooth numbered 6, the upper right tooth numbered 5, and an upper right tooth numbered 4 appear, in this order from the left. Accordingly, in the tooth number display areas 204 (see a reference numeral H4) displayed above the upper left second image of the image display area group 213, the numbers 7, 6, 5, and 4 are displayed in this order from the left.

In this way, at least a part of teeth included in two images arranged and displayed adjacently among a plurality of images arranged and displayed in the image display area group 213 overlap each other.

In the X-ray image presence/absence selection area 205, whether a corresponding X-ray image is valid or invalid is selected by clicking. Note that the X-ray image presence/absence selection area 205 may be simply referred to as the selection area 205.

In the input screen 200 illustrated in FIG. 4, all X-ray images are set valid. When the upper left first selection area 205 (see the reference numeral H2) of the input screen 200 is clicked and the X-ray image is set invalid, the upper left first evaluation result input areas 206 (see the reference numeral H2) of the input screen 200 and the upper left first X-ray image (see the reference numeral H1) of the image display area group 213 are hidden.

In a case where evaluation results have been input in the evaluation result input areas 206 when the X-ray image is set invalid in the selection area 205, the input evaluation results may be held without being deleted.

In the evaluation result input areas 206, a user inputs evaluation results of corresponding teeth. Note that the evaluation result input areas 206 may be simply referred to as the input areas 206.

Each of the evaluation results is displayed by, for example, G, Q, H, X, N or I. For example, G indicates a tooth that is in a good condition and is less likely to fall out within a predetermined period (for example, within 10 years), Q indicates a tooth for which it is difficult to determine whether or not the tooth falls out within the predetermined period, and H indicates a tooth that is likely to fall out within the predetermined period. Furthermore, for example, X indicates a missing tooth, N indicates a tooth for which it is difficult to make determination because a part of the tooth does not appear or is unclear in an X-ray image, and I indicates a tooth implant.

The evaluation result displayed in the input areas 206 may be switched by clicking the corresponding number in the tooth number display areas 204. For example, the evaluation result may be switched in the order of G, Q, H, X, N, and I every time the corresponding number in the tooth number display areas 204 is left-clicked, and may be switched in the order of I, N, X, H, Q, and G every time the corresponding number in the tooth number display areas 204 is right-clicked.

Furthermore, the evaluation results displayed in the input area 206 may be input by G, Q, H, X, N, and I keys on the keyboard 152 of the terminal 2. In a case where the evaluation result displayed in the input areas 206 is input in a lowercase alphabet, the lowercase alphabet may be automatically converted into an uppercase alphabet.

Moreover, the evaluation results displayed in the input areas 206 may be input by causing an optional numeric key or the like on the keyboard 152 of the terminal 2 to function as a shortcut key.

In a case where the evaluation result is input by the keyboard 152 of the terminal 2, the input processing unit 114 automatically moves an input cursor displayed in one box of the input areas 206 to the next box. In addition, in each set of the plurality of input areas 206 of the input screen 200, the input processing unit 114 moves the input cursor in a substantially “U” shape (for example, “shape in which U is turned over to the left”).

For example, when information indicating a condition of each tooth is input in corresponding one of the plurality of input areas 206, the input processing unit 114 moves the input cursor displayed in the corresponding input area 206 in a direction from an upper right molar to an upper left molar in a tooth arrangement order. Thereafter, the input processing unit 114 moves the input cursor displayed in the corresponding input area 206 in a direction from a lower left molar to a lower right molar in the tooth arrangement order.

As described above, at least a part of teeth included in two images arranged and displayed adjacently among a plurality of images arranged and displayed in the image display area group 213 overlap each other. Thus, in a case where evaluation results input in two or more input areas 206 corresponding to the same tooth are different from each other, a user may be warned that there is a discrepancy in the evaluation results by displaying the corresponding tooth number in red, for example. However, the user need not be warned in a case where one of the different evaluation results is “N” indicating a tooth for which it is difficult to make determination because a part of the tooth does not appear or is unclear in an X-ray image.

For example, in a case where there is a mismatch in pieces of information which are input in input areas corresponding to the two images and indicate conditions of a part of teeth overlapping each other, the input processing unit 114 notifies the mismatch. Furthermore, in a case where any one of the pieces of information which are input in the input areas 206 corresponding to the two images and indicate conditions of a part of teeth overlapping each other indicates that it is difficult to determine the conditions of the teeth, the input processing unit 114 inhibits the notification of the mismatch.

When the batch input button 207 is clicked, an evaluation result set in advance by a user is input to all the input areas 206 or values already input are cleared from all the input areas 206. Furthermore, when the batch input button 207 is clicked, an X-ray image set in advance by a user may be set invalid. In the illustrated example, the batch input button 207 includes buttons “I” to “V” and “C”.

For example, when the “I” button is clicked, the evaluation result “G” may be input in all the input areas 206. Furthermore, for example, when the “II” button is clicked, some X-ray images may be set invalid. Moreover, for example, when the “C” button is clicked, the evaluation results input in all the input areas 206 may be cleared.

When the cancel button 208 is clicked, evaluation results input in all the input areas 206 and the X-ray images displayed in the image display area group 213 may be cleared. For the cancel button 208, as illustrated in FIG. 4, “F1” key on the keyboard 152 of the terminal 2 may be set as a shortcut key.

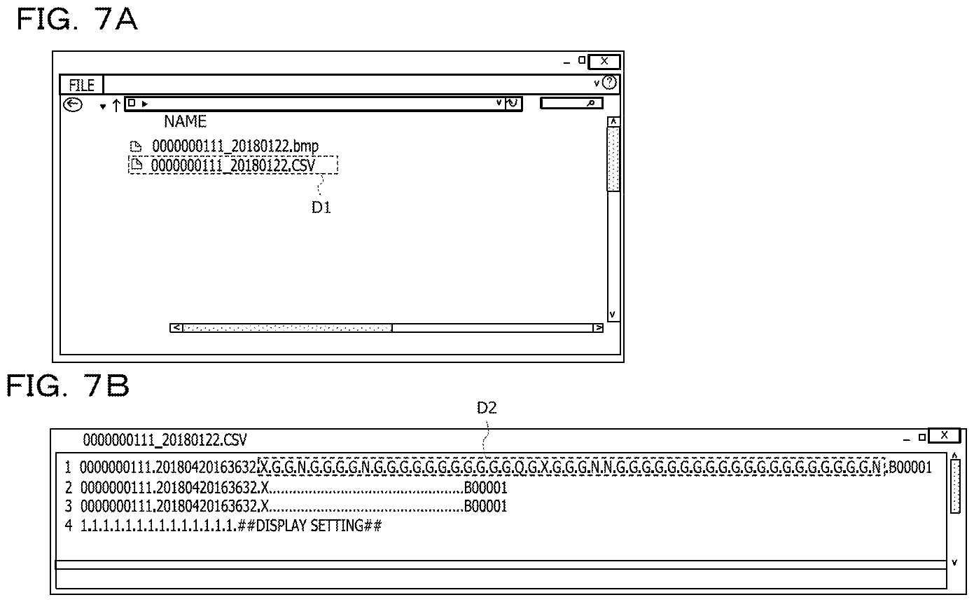

When the temporary storage button 209 is clicked, evaluation results that have been already input are stored in association with tooth numbers. For the temporary storage button 209, as illustrated in FIG. 4, “F5” key on the keyboard 152 of the terminal 2 may be set as a shortcut key. Note that a configuration example of the temporary storage data will be described later with reference to FIG. 7.

When the automatic input button 210 is clicked, evaluation results are automatically input in the input areas 206 on the basis of past learning data. For the automatic input button 210, as illustrated in FIG. 4, “F8” key on the keyboard 152 of the terminal 2 may be set as a shortcut key.

For example, in a case where there is a learning model generated by the generation processing unit 115, the input processing unit 114 automatically inputs information indicating a condition of each tooth in each of the plurality of input areas 206 using the generated learning model.

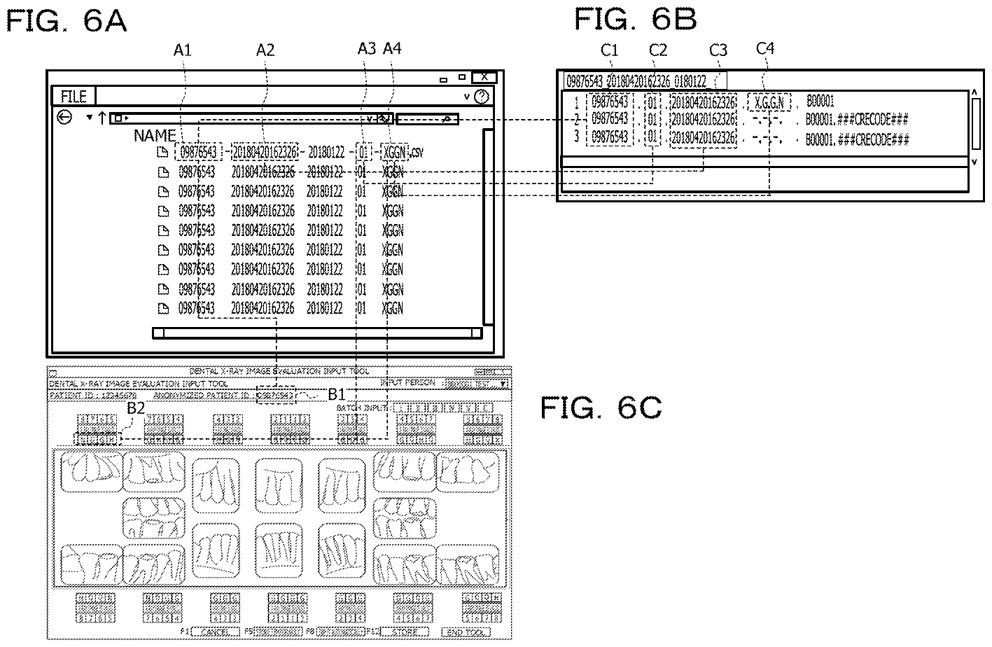

When the storage button 211 is clicked, evaluation results in all the input areas 206 are stored in association with tooth numbers. For the storage button 211, as illustrated in FIG. 4, “F12” key on the keyboard 152 of the terminal 2 may be set as a shortcut key. Note that a configuration example of the storage data will be described later with reference to FIG. 6.

In a case where the storage button 211 is clicked when there is one or more input areas 206 in which evaluation results have not been input, for example, a pop-up window may be displayed for inquiring a user whether evaluation results may be collectively set to “G” in the input areas 206 in which evaluation results have not been input.

When the tool end button 212 is clicked, the input screen 200 is hidden, and the dental X-ray image evaluation tool ends.

A plurality of X-ray images is displayed in the image display area group 213. In the example illustrated in FIG. 4, up to 16 X-ray images are displayed. Note that, among the 16 X-ray images, two X-ray images indicated by reference numerals H5 and H6 are auxiliary images for observing bite of upper and lower teeth.

When the dental X-ray image evaluation tool is activated, X-ray images are not displayed in the image display area group 213, and for example, X-ray images of an optional patient may become selectable by clicking the image display area group 213. Furthermore, in a case where the image display area group 213 is clicked after X-ray images are selected, X-ray images of the next patient may become selectable. Moreover, X-ray images of the next patient may become selectable by dragging and dropping an icon of X-ray image data to the image display area group 213.

The generation processing unit 115 may calculate evaluation values of teeth (for example, “oral cavity”) for each patient. In this case, a tooth with the evaluation result H, X, or I may not be evaluated, and a tooth with the evaluation result G or Q indicating that the tooth is healthy or has room for improvement may be evaluated.

The evaluation value may be calculated, for example, by the following formula.

Evaluation value=xg−ag+w*(xq−aq)

Here, ag indicates the standard number of teeth indicating the evaluation result G, aq indicates the standard number of teeth indicating the evaluation result Q, xg indicates the number of teeth to be evaluated indicating the evaluation result G, and xq indicates the number of teeth to be evaluated indicating the evaluation result Q. Furthermore, w is a weighted value of the number of teeth indicating the evaluation result Q relative to the number of teeth indicating the evaluation result G, and is an optional value of 0 or more and less than 1.

FIG. 5 is a diagram illustrating a second screen display example in the terminal 2 illustrated in FIG. 2.

In the input screen 200 illustrated in FIG. 5, display of X-ray images is set to be hidden in the selection areas 205 denoted by reference numerals I1 to I8. As a result, the input areas 206 and X-ray images corresponding to the selection areas 205 denoted by the reference numerals I1 to I8 are not displayed.

In a case where X-ray images of the next patient are selected, selection contents of the selection areas 205 may be held. For example, in a case where X-ray images of the next patient are selected in the state illustrated in FIG. 5, display of X-ray images is continuously set to be hidden in the selection area 205 denoted by the reference numerals I1 to I8.

For example, in a case where a plurality of images different from a plurality of images is newly acquired, the input processing unit 114 holds, in the selection areas 205 for the different plurality of images, selection contents for the plurality of images.

FIGS. 6A to 6C are diagrams illustrating a configuration example of storage data in the terminal 2 illustrated in FIG. 2. Specifically, FIG. 6A illustrates a list of files of storage data, FIG. 6B illustrates display contents stored in the input screen 200, and FIG. 6C illustrates contents of the storage data in a comma-separated values (CSV) format.

In the list of the files illustrated in FIG. 6A, a plurality of files in the CSV format is displayed for each input area 206 in the input screen 200.

In the contents of the storage data illustrated in FIG. 6C, an evaluation result of each tooth is registered in a first line in association with other values. Second and third lines are expansion areas, in which, for example, information regarding a degree of plaque adhesion on each tooth and the depth of a periodontal pocket may be registered.

“09876543” denoted by a reference numeral A1 of a file name in FIG. 6A and a reference numeral C1 of the contents of the storage data in FIG. 6C corresponds to an anonymized patient ID denoted by a reference numeral B1 in FIG. 6B.

“20180420162326” denoted by a reference numeral A2 of the file name in FIG. 6A and a reference numeral C2 of the contents of the storage data in FIG. 6C indicates 16:23:26 on Apr. 20, 2018, which is date and time when evaluation results were registered in FIG. 6B.

“01” denoted by a reference numeral A3 of the file name in FIG. 6A and a reference numeral C3 of the contents of the storage data in FIG. 6C indicates a position of the input areas 206 denoted by a reference numeral B2 in FIG. 6B. The positions of the input areas 206 are 01, 02, 03, 04, 05, 06, and 07 in this order from the upper left to the upper right of the input screen 200, and are 08, 09, 10, 11, 12, 13, and 14 in this order from the lower left to the lower right of the input screen 200.

“XGGN” denoted by a reference numeral A4 of the file name in FIG. 6A and “X, G, G, N” denoted by a reference numeral C4 of the contents of the storage data in FIG. 6C indicates evaluation results in the input areas 206 denoted by the reference numeral B2 in FIG. 6B. By registering evaluation results in a file name in this way, a processing speed in AI analysis processing may be improved. As an evaluation result of a tooth for which an X-ray image is set invalid in the selection area 205, “-” or “0” may be registered.

In the storage data, information such as age, sex, and smoking history of a patient may be registered.

FIGS. 7A and 7B are diagrams illustrating a configuration example of temporary storage data in the terminal 2 illustrated in FIG. 2. Specifically, FIG. 7A illustrates a list of files of temporary storage data, and FIG. 7B illustrates contents of the temporary storage data in the CSV format.

As illustrated in FIG. 7A, for example, in a case where evaluation results are temporarily stored, image data in a bitmap (BMP) format that consolidates a plurality of X-ray images of the patient and data in the CSV format including the evaluation results and the like that have already been input are stored in the same folder.

The temporary storage data illustrated in FIG. 7B is displayed by selecting the file denoted by a reference numeral D1 in FIG. 7A. In the temporary storage data, all the evaluation results that have already been input are consolidated and registered, as denoted by a reference numeral D2 in FIG. 7B

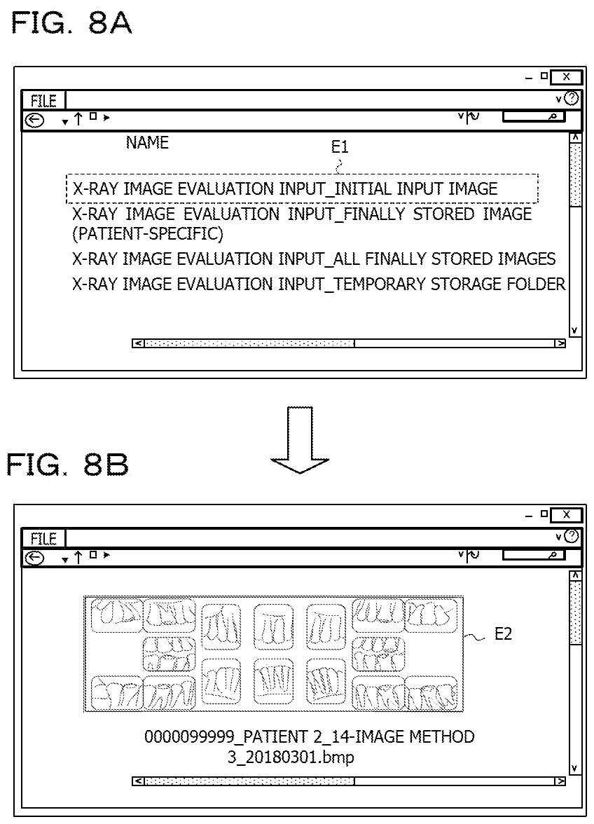

FIGS. 8A and 8B are diagrams illustrating a configuration example of image data for which evaluation results are not input in the terminal 2 illustrated in FIG. 2. Specifically, FIG. 8A illustrates a list of image storage folders, and FIG. 8B illustrates a preview image of image data for which evaluation results are not input.

When a folder of “X-ray image evaluation input_initial input image” (see a reference numeral E1) is selected as illustrated in FIG. 8A, the image data for which evaluation results are not input (see a reference numeral E2) is displayed as illustrated in FIG. 8B. As illustrated in FIG. 8B, in an initial state where the evaluation results are not input, image data in the BMP format that consolidates a plurality of X-ray images of the patient is stored.

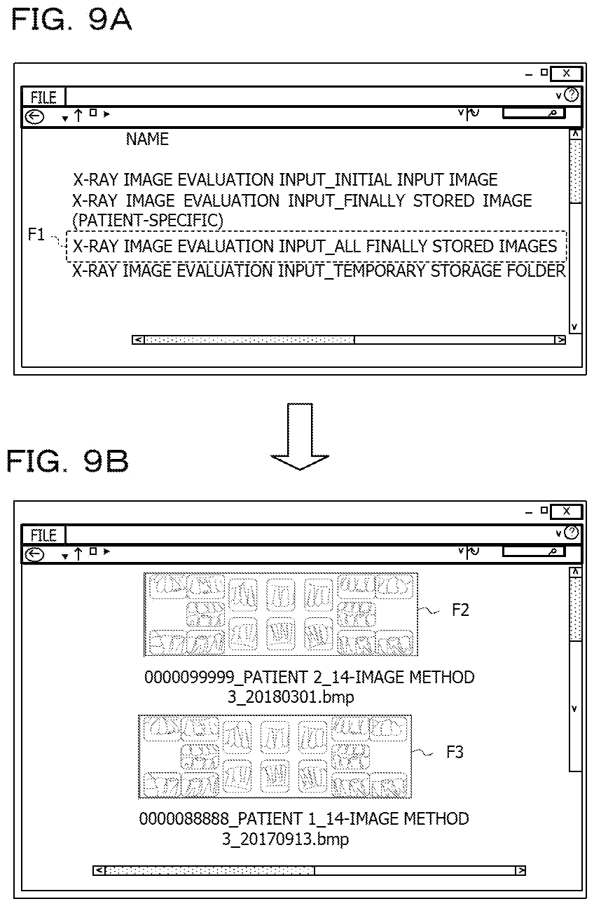

FIGS. 9A and 9B are diagrams illustrating a configuration example of all stored image data in the terminal 2 illustrated in FIG. 2. Specifically, FIG. 9A illustrates a list of image storage folders, and FIG. 9B illustrates preview images of stored image data.

When a folder of “X-ray image evaluation input_all finally stored images” (see a reference numeral F1) is selected as illustrated in FIG. 9A, a list of all finally stored image data (see reference numerals F2 and F3) is displayed as illustrated in FIG. 9B. As illustrated in FIG. 9B, as the finally stored image data, image data in the BMP format that consolidates a plurality of X-ray images of the patient is stored.

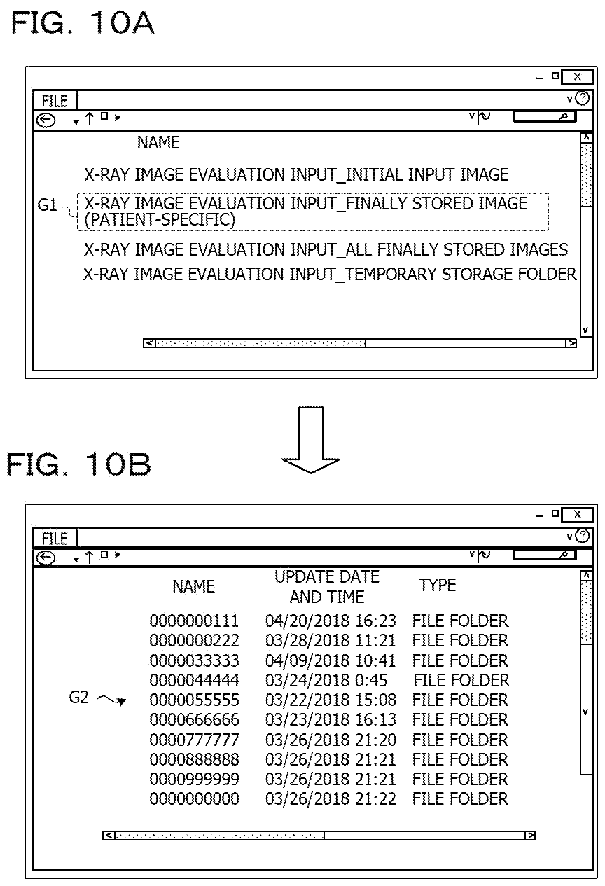

FIGS. 10A and 10B are diagrams illustrating a configuration example of stored patient-specific image data in the terminal 2 illustrated in FIG. 2. Specifically, FIG. 10A illustrates a list of image storage folders, and FIG. 10B illustrates a list of folders in which stored image data is stored.

When a folder of “X-ray image evaluation input_finally stored image (patient-specific)” (see a reference numeral G1) is selected as illustrated in FIG. 10A, the list of folders in which finally stored image data is stored for each patient (see a reference numeral G2) is displayed as illustrated in FIG. 10B. In each folder, the stored patient-specific image data may be divided into a plurality of images and stored.

[A-2] Operation Example

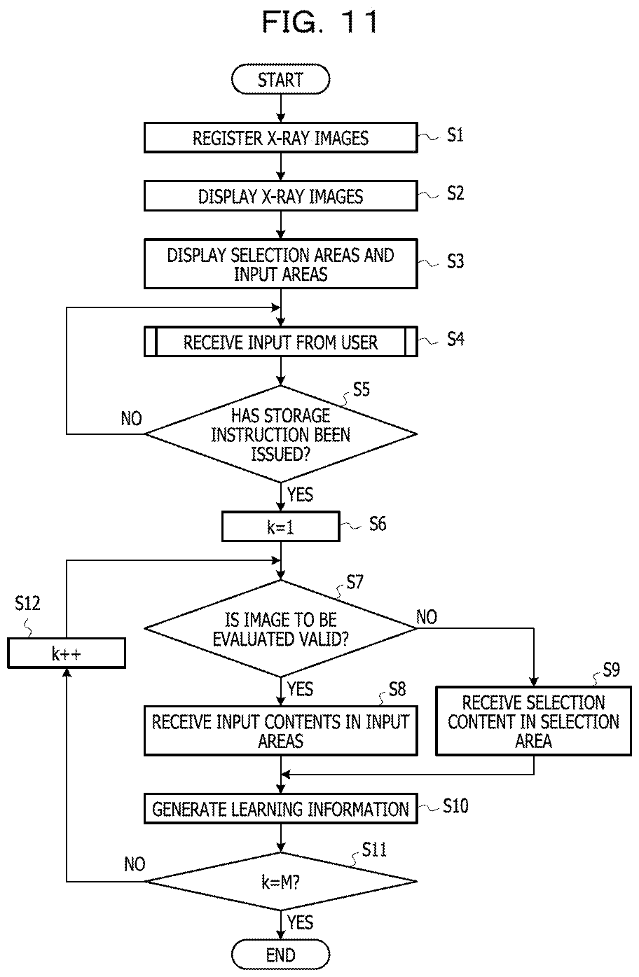

With reference to a flowchart (Steps S1 to S12) illustrated in FIG. 11, learning information generation processing in the information processing apparatus 1 illustrated in FIG. 2 will be described.

The acquisition processing unit 111 registers, in response to a request from the terminal 2, a plurality of X-ray images as X-ray images to be evaluated (Step S1).

In the terminal 2, the first display processing unit 112 displays the registered X-ray images on the input screen 200 (Step S2).

In the terminal 2, the second display processing unit 113 displays the selection areas 205 and the input areas 206 on the input screen 200 (Step S3).

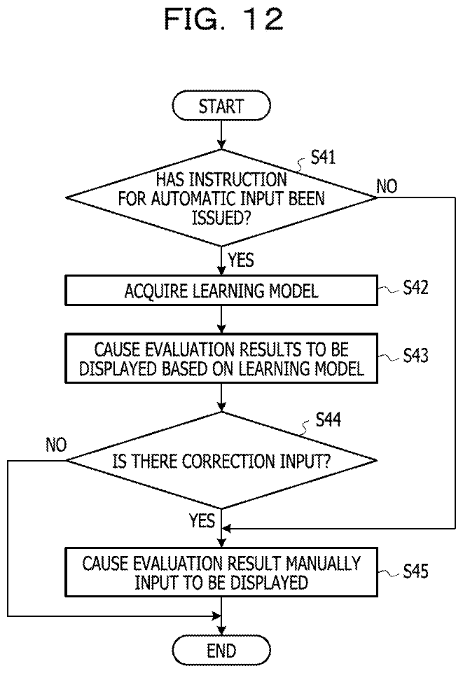

The input processing unit 114 receives input from a user to the selection areas 205 or the input areas 206 (Step S4). Note that details of the processing in Step S4 will be described later with reference to FIG. 12.

When the storage button 211 is clicked, the input processing unit 114 determines whether or not a storage instruction of evaluation results has been issued (Step S5).

In a case where the storage instruction has not been issued (see a No route in Step S5), the processing returns to Step S4.

On the other hand, in a case where the storage instruction has been issued (see a Yes route in Step S5), the input processing unit 114 sets a variable k to 1 (Step S6).

The input processing unit 114 determines whether a k-th X-ray image to be evaluated is set to be valid in the selection area 205 (Step S7).

In a case where the X-ray image to be evaluated is set to be valid (see a Yes route in Step S7), the input processing unit 114 receives input contents in the input areas 206 (Step S8). Then, the processing proceeds to step S10.

On the other hand, in a case where the X-ray image to be evaluated is not set to be valid (see a No route in Step S7), the input processing unit 114 receives a selection content indicating invalidity of the X-ray image in the selection area 205 (Step S9).

The generation processing unit 115 generates learning information based on the input contents in the input areas 206 or the selection content in the selection area 205 (Step S10).

The input processing unit 114 determines whether the variable k has reached the number M of the X-ray images set to be valid (Step S11).

In a case where the variable k has not reached M (see a No route in Step S11), the input processing unit 114 increments the variable k by “1” (Step S12). Then, the processing returns to step S7.

On the other hand, in a case where the variable k has reached M (see a Yes route in Step S11), the learning information generation processing ends.

Next, the details of the input reception processing (Step S4) illustrated in FIG. 11 will be described with reference to a flowchart (steps S41 to S45) illustrated in FIG. 12.

When the automatic input button 210 is clicked, the input processing unit 114 determines whether an instruction for automatic input has been issued (Step S41).

In a case where the instruction for automatic input has not been issued (see a No route in Step S41), the processing proceeds to Step S45.

On the other hand, in a case where the instruction for automatic input has been issued (see a Yes route in Step S41), the input processing unit 114 acquires a learning model (Step S42).

In the terminal 2, the input processing unit 114 causes an evaluation result in each input area 206 to be displayed on the input screen 200, on the basis of the acquired learning model (Step S43).

The input processing unit 114 determines whether there is correction input from a user for automatically input evaluation results (Step S44).

In a case where there is no correction input (see a No route in Step S44), the input reception processing ends.

On the other hand, in a case where there is correction input (see a Yes route in Step S44), in the terminal 2, the input processing unit 114 causes an evaluation result manually input by the user to be displayed on the input screen 200 (Step S45). Then, the input reception processing ends.

[A-3] Effect

According to the information processing apparatus 1 in the example of the embodiment described above, for example, the following effects may be obtained.

The acquisition processing unit 111 acquires a plurality of images of N or less each including a plurality of teeth adjacent to each other. In N image display areas 213 capable of displaying a plurality of images in association with an arrangement of teeth, the first display processing unit 112 arranges and displays each of the plurality of images in corresponding one of the image display areas 213. The second display processing unit 113 displays, in association with each of the N image display areas 213, the selection area 205 for selecting presence or absence of an image and the plurality of input areas 206 for inputting information indicating a condition of each tooth included in an image displayed in each of the N image display areas 213. The input processing unit 114 receives at least one of a selection content in the selection area 205 and input contents in the plurality of input areas 206, corresponding to each of the N image display areas 213. The generation processing unit 115 generates learning information in which a condition of each of a plurality of teeth and an image of each of the plurality of teeth are associated with each other in a state where positions of the plurality of teeth may be identified, on the basis of the received selection contents and input contents, and the registered plurality of images.

With this configuration, learning data for automatic diagnosis of a condition of an oral cavity may be efficiently generated. Furthermore, since values of evaluation results may be unified and a user interface (UI) adapted to thoughts and habits of a dentist or a dental hygienist is provided, it is possible to input position information and an evaluation result of a tooth with minimum labor and time.

The generation processing unit 115 uses the generated learning information to generate, by deep learning, a learning model which relates to characteristics corresponding to a condition of each tooth. The input processing unit 114 uses the generated learning model to generate information indicating a condition of each of a plurality of teeth included in a new image. Then, the second display processing unit 113 outputs the information indicating the condition of each of the plurality of teeth included in the new image.

With this configuration, a condition of each tooth may be automatically evaluated on the basis of the learning model.

When information indicating a condition of each tooth is input in corresponding one of the plurality of input areas 206, the input processing unit 114 moves an input cursor displayed in the corresponding input area 206 in a direction from an upper right molar to an upper left molar in a tooth arrangement order. Thereafter, the input processing unit 114 moves the input cursor displayed in the corresponding input area 206 in a direction from a lower left molar to a lower right molar in the tooth arrangement order.

With this configuration, since the input order of an evaluation result of each tooth matches a general diagnosis order of each tooth by a dentist, the evaluation result of each tooth may be input efficiently.

At least a part of teeth included in two images arranged and displayed adjacently among the plurality of images arranged and displayed overlap each other. In a case where there is a mismatch in pieces of information which are input in input areas corresponding to the two images and indicate conditions of the part of teeth overlapping each other, the input processing unit 114 notifies the mismatch.

With this configuration, it is possible to reduce discrepancy in evaluation results for the same tooth.

In a case where any one of the pieces of information which are input in the input areas 206 corresponding to the two images and indicate conditions of the part of teeth overlapping each other indicates that it is difficult to determine the conditions of the teeth, the input processing unit 114 inhibits the notification.

With this configuration, it is possible to prevent an alert from being issued in a case where, for example, an evaluation result “G” indicating a good tooth is input in the input area 206 corresponding to one of the two images, and an evaluation result “N” indicating that it is difficult to perform evaluation because the image is unclear is input in the input area 206 corresponding to the other image.

In a case where a plurality of images different from a plurality of images is newly acquired, the input processing unit 114 holds, in the selection areas 205 for the different plurality of images, selection contents for the plurality of images.

With this configuration, when evaluation results of teeth are entered for a second patient after evaluation results of teeth are entered for a first patient, in a case where remaining positions of the teeth of the first patient and the second patient are similar, the evaluation results may be input efficiently. For example, since probability that a molar remains is low in an elderly person, an input operation may be efficiently performed when evaluation results for a plurality of elderly persons are sequentially input.

[B] Others

The disclosed technique is not limited to the embodiment described above, and various modifications may be made without departing from the spirit of the present embodiment. Each configuration and each processing of the present embodiment may be selected or omitted as needed or may be appropriately combined.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium having stored therein a program for causing a computer to execute processing comprising:
acquiring a plurality of images of N or less each including a plurality of teeth adjacent to each other;
arranging and displaying, in N image display areas capable of displaying the plurality of images in association with an arrangement of teeth, each of the plurality of images in corresponding one of the image display areas;
displaying, in association with each of the N image display areas, a selection area for selecting presence or absence of an image and a plurality of input areas for inputting information indicating a condition of each tooth included in an image displayed in each of the N image display areas;
receiving a selection content in the selection area or input contents in the plurality of input areas or combination of the selection content and the input contents, corresponding to each of the N image display areas; and
generating learning information in which a condition of each of the plurality of teeth and an image of each of the plurality of teeth are associated with each other in a state capable of identifying positions of the plurality of teeth, on the basis of the received selection content and input contents and the plurality of images that has been registered.

2. The non-transitory computer-readable recording medium according to claim 1, for causing the computer to execute processing comprising:
generating, by deep learning, a learning model which corresponds to each of the plurality of teeth and relates to characteristics regarding the condition of each tooth, using the generated learning information;
generating, using the generated learning model, information indicating a condition of each of a plurality of teeth included in a new image; and
outputting the information indicating the condition of each of the plurality of teeth included in the new image.

3. The non-transitory computer-readable recording medium according to claim 1, for causing the computer to execute processing comprising
when the information indicating the condition is input in the plurality of input areas, moving an input cursor displayed in corresponding one of the input areas in a direction from an upper right molar to an upper left molar in a tooth arrangement order, and moving the input cursor displayed in corresponding one of the input areas in a direction from a lower left molar to a lower right molar in the tooth arrangement order.

4. The non-transitory computer-readable recording medium according to claim 1, for causing the computer to execute processing comprising
in a case where at least a part of teeth included in two images arranged and displayed adjacently among the plurality of images arranged and displayed overlap each other, and
there is a mismatch in pieces of the information which are input in input areas corresponding to the two images and indicate conditions of the part of the teeth overlapping each other,
notifying the mismatch.

5. The non-transitory computer-readable recording medium according to claim 4, for causing the computer to execute processing comprising
inhibiting the notification in a case where any one of the pieces of the information which are input in the input areas corresponding to the two images and indicate the conditions of the part of the teeth overlapping each other indicates that it is difficult to determine the conditions of the teeth.

6. The non-transitory computer-readable recording medium according to claim 1, for causing the computer to execute processing comprising
in a case where a plurality of images different from the plurality of images is newly acquired, holding, in selection areas for the different plurality of images, the selection contents for the plurality of images.

7. An information processing apparatus comprising:
a memory; and
a processor coupled to the memory and configured to:
acquire a plurality of images of N or less each including a plurality of teeth adjacent to each other;
arrange and displays, in N image display areas capable of displaying the plurality of images in association with an arrangement of teeth, each of the plurality of images in corresponding one of the image display areas;
display, in association with each of the N image display areas, a selection area for selecting presence or absence of an image and a plurality of input areas for inputting information indicating a condition of each tooth included in an image displayed in each of the N image display areas;
receive a selection content in the selection area or input contents in the plurality of input areas or combination of the selection content and the input contents, corresponding to each of the N image display areas; and
generate learning information in which a condition of each of the plurality of teeth and an image of each of the plurality of teeth are associated with each other in a state capable of identifying positions of the plurality of teeth, on the basis of the received selection content and input contents, and the plurality of images that has been registered.

8. The information processing apparatus according to claim 7, wherein the processor is configured to:
generate, by deep learning, a learning model which corresponds to each of the plurality of teeth and relates to characteristics regarding the condition of each tooth, using the generated learning information,
generate, using the learning model, information indicating a condition of each of a plurality of teeth included in a new image, and
output the information indicating the condition of each of the plurality of teeth included in the new image.

9. The information processing apparatus according to claim 7, wherein the processor is configured to:
when the information indicating the condition is input in the plurality of input areas, move an input cursor displayed in corresponding one of the input areas in a direction from an upper right molar to an upper left molar in a tooth arrangement order, and
move the input cursor displayed in corresponding one of the input areas in a direction from a lower left molar to a lower right molar in the tooth arrangement order.

10. The information processing apparatus according to claim 7, wherein
in a case where at least a part of teeth included in two images arranged and displayed adjacently among the plurality of images arranged and displayed overlap each other, and
there is a mismatch in pieces of the information which are input in input areas corresponding to the two images and indicate conditions of the part of the teeth overlapping each other, the processor notifies the mismatch.

11. The information processing apparatus according to claim 10, wherein the processor is configured to:
inhibit the notification in a case where any one of the pieces of the information which are input in the input areas corresponding to the two images and indicate conditions of the part of the teeth overlapping each other indicates that it is difficult to determine the conditions of the teeth.

12. The information processing apparatus according to claim 7, wherein
in a case where a plurality of images different from the plurality of images is newly acquired, the selection contents in the plurality of images are held in selection areas for the different plurality of images.

13. A method for processing information, the method comprising executing, by a computer, processing comprising:
acquiring a plurality of images of N or less each including a plurality of teeth adjacent to each other;
arranging and displaying, in N image display areas capable of displaying the plurality of images in association with an arrangement of teeth, each of the plurality of images in corresponding one of the image display areas;
displaying, in association with each of the N image display areas, a selection area for selecting presence or absence of an image, and a plurality of input areas for inputting information indicating a condition of each tooth included in an image displayed in each of the N image display areas;
receiving a selection content in the selection area or input contents in the plurality of input areas or combination of the selection content and the input contents, corresponding to each of the N image display areas; and
generating learning information in which a condition of each of the plurality of teeth and an image of each of the plurality of teeth are associated with each other in a state capable of identifying positions of the plurality of teeth, on the basis of the received selection content and input contents, and the plurality of images that has been registered.

14. The method according to claim 13, further comprising:
generating, by deep learning, a learning model which corresponds to each of the plurality of teeth and relates to characteristics regarding the condition of each tooth, using the generated learning information;
generating, using the generated learning model, information indicating a condition of each of a plurality of teeth included in a new image; and
outputting the information indicating the condition of each of the plurality of teeth included in the new image.

15. The method according to claim 13, further comprising
when the information indicating the condition is input in the plurality of input areas, moving an input cursor displayed in corresponding one of the input areas in a direction from an upper right molar to an upper left molar in a tooth arrangement order, and moving the input cursor displayed in corresponding one of the input areas in a direction from a lower left molar to a lower right molar in the tooth arrangement order.

16. The method according to claim 13, further comprising
in a case where at least a part of teeth included in two images arranged and displayed adjacently among the plurality of images arranged and displayed overlap each other, and
there is a mismatch in pieces of the information which are input in input areas corresponding to the two images and indicate conditions of the part of the teeth overlapping each other,
notifying the mismatch.

17. The method according to claim 16, further comprising
inhibiting the notification in a case where any one of the pieces of the information which are input in the input areas corresponding to the two image and indicate the conditions of the part of the teeth overlapping each other indicates that it is difficult to determine the conditions of the teeth.

18. The method according to claim 13, further comprising
in a case where a plurality of images different from the plurality of images is newly acquired, holding, in selection areas for the different plurality of images, the selection contents in the plurality of images.
